(19) United States
(12) Patent Application Publication
(10) Pub. No.: US 2012/0164756 A1
(43) Pub. Date: Jun. 28, 2012

(54) ASPIRIN ASSAY

(75) Inventors: Elouard Benchikh, Crumlin (GB); Stephen Peter Fitzgerald, Crumlin (GB); Philip Andrew Lowry, Crumlin (GB); Paul John Innocenzi, Crumlin (GB); Ivan Robert McConnell, Crumlin (GB)

(73) Assignee: RANDOX LABORATORIES LIMITED, Crumlin (GB)

(21) Appl. No.: 13/389,373

(22) PCT Filed: Jun. 9, 2010
(86) PCT No.: PCT/GB2010/050962

§ 371 Date: Feb. 7, 2012

(30) Foreign Application Priority Data
Jun. 10, 2009 (GB) 0909925.0

(51) Int. Cl. G01N 33/53; C07K 16/00

(52) U.S. Cl. 436/501; 530/389.8

(57) ABSTRACT

The invention describes a method for monitoring and detecting non-therapeutic, therapeutic and toxic concentrations of aspirin in individuals which uses the urinary salicylic acid to salicyluric acid ratio.

### FIELD OF THE INVENTION

The invention relates to improved methods for the monitoring and quantification of aspirin levels for the purposes of therapeutic drug monitoring and overdose incidents using the ratio of salicylic acid to salicyluric acid in urine. The methods and kits described optionally incorporate a novel salicyluric acid-specific antibody.

### BACKGROUND OF THE INVENTION

Aspirin (common scientific name acetylsalicylic acid, IUPAC name 2-acetoxybenzoic acid), is one of the most used drugs, possessing analgesic, anti-pyretic and anti-inflammatory properties. In addition to its common use as a treatment for fever and pain, it is clinically used to treat stroke, myocardial infarction, angina, colorectal cancer and rheumatoid arthritis. Its effectiveness as a therapeutic drug is constrained by a narrow concentration range which in serum is a level of approximately 150-300 mg/l. Concentrations below this level are likely to be sub-therapeutic, while concentrations exceeding 300 mg/l are considered to be toxic.

The pharmacokinetics of aspirin have been studied in detail. Ingestion of a therapeutic amount of aspirin is followed by rapid hydrolysis to salicylic acid (SA) with peak levels of salicylic acid occurring after 1-2 hours. Within 2 hours only trace levels of aspirin remain. The half-life of salicylic acid is approximately 6 hours in serum, its hepatic metabolism following several pathways. The principal salicylic acid metabolite is the glycine conjugate salicyluric acid (SUA) which makes up approximately 75% of excreted product. The other main excretory products are gentisic acid (GA—approximately 1%), salicylphenolic glucuronide (SPG—approximately 8%) and salicylacyl glucuronide (SAG—approximately 5%). Unchanged salicylic acid is excreted at a level of approximately 10%. Formation of SUA and SPG follow Michaelis-Menten or zero-order kinetics, while SAG and GA follow first-order kinetics. This means that as the amount of ingested aspirin increases, the SUA and SPG pathways become saturated and the amount excreted levels off leading to an increase in the amount of product excreted as GA, SAG and especially SA.

The availability of aspirin in numerous over-the-counter products and its wide-spread therapeutic use means that aspirin is commonly found in unwell and drug overdose patients presenting at hospital emergency departments. The overdose patients can be categorised as intentional and accidental, the latter often represented by young children and the elderly. There are several methods for monitoring aspirin therapy and determining overdose such as high performance liquid chromatography (HPLC), liquid chromatography-mass spectrometry (LC-MS) or spectrophotometric methods using enzymes and complexation reagents. These methods have drawbacks such as being non-specific, having lengthy assay times, requiring expensive equipment requirements and specialist operators (HPLC/LC-MS), and requiring time-consuming pre-treatment steps. For example, the enzymatic and complexation assays are usually for use with either serum or plasma and due to their sensitivity are usually inadequate for overdose (OD) determination.

An alternative method for testing for aspirin OD is the use of the immunoassay. The benefits of the immunoassay are manifold, especially its simplicity of execution and affordability. Several immunoassays for salicylate detection have been reported.

EP 0194352 refers to a salicylate fluorescence polarization immunoassay; U.S. Pat. No. 5,089,388 describes an enzymatic immunoassay which detects SA, ASA, GA, 2,3-dihydroxybenzoic acid and methyl salicylate. A lateral flow immunoassay which detects both paracetamol and aspirin has been described (Song and Dou 2003). An EMIT immunoassay for salicylic acid has also been described (Fraser 1983). The current immunoassays measure the ‘salicylate’ level in serum or plasma. Historically, salicylate referred to salicylic acid although current use of the term generally refers to one or more aspirin metabolites. These metabolites include SA, SUA, SPG, SAG and GA. Aspirin overdose can be defined clinically using a semi-quantitative immunoassay format which measures the serum concentration of salicylates at a defined cut-off concentration. The immunoassays available have several drawbacks such as their non-specific nature (the antibody identifies several metabolites which makes quantitation less precise), sample pre-treatment requirements and, most notably, errors associated with calculations to quantify aspirin concentration (unit conversions and dilution correction calculations). It is also proposed that serum salicylate levels frequently do not reflect the severity of the poisoning (O'Malley 2007). Furthermore, serum testing is invasive. A urinary-based assay would be less invasive and simpler, ideally not requiring a pre-assay sample preparation step.

Aspirin overdose patients usually present at a hospital several hours after drug ingestion in a conscious state, so metabolite detection and determination using a ‘non-invasive’ urine sample would appear to be a more convenient sample type for testing than blood. If the patient presents in an unconscious state a catheter can be implemented. Use of blood as sample type for salicylate testing may have a historical basis. Done's nomogram concept for aiding in the assessment of drug toxicity based on time of ingestion was originally based on plasma salicylate levels. The Done nomogram concept is now recognised as not being of use in cases of sub-acute and chronic salicylate intoxication (Casarett and Doull's Toxicology, 6^{th} Edition). It has been suggested that if plasma salicylate concentration is to be used in such instances, measurements should be taken every three to four hours until the plasma concentration peaks, as the plasma salicylate concentration at presentation can be an unreliable guide to the severity of poisoning (Greene et al. 2005). Of particular concern regarding current blood-based assays is the occurrence of errors in the calculation of salicylate levels from a suspected overdose patient (Hahn et al. 2000; Goldfrank's Toxicologic Emergencies 2002; Eldridge and Holstege 2006; O'Malley 2007). Errors related to dilution correction calculations and to unit conversions for concentration levels can result in misdiagnosis with potentially fatal consequences for an overdose patient.

Patients presenting with aspirin poisoning can be the young, the elderly, self-medicating individuals or individuals on long-term aspirin therapy. Cases of chronic aspirin poisoning in the elderly can partly be a result of the slower and less regular metabolism associated with older individuals and partly due to the higher likelihood of ingestion of the wrong dose or of additional doses of aspirin. Conversely, individuals on aspirin therapy may be under self-medicating and thus unknowingly be prone to the disease or condition for which they assume they have protection. A solution to the problem of aspirin under-dosing or poisoning in individuals self-medicating or on long-term therapy would be for a self-monitoring or doctor's test to ensure that aspirin is at therapeutic and non-toxic levels. Current salicylate tests cannot meet this need as a blood sample is required.

Taking into account the current short-comings of existing tests for aspirin overdose and therapeutic drug monitoring, a new assay which circumvents these problems is desirable.

### SUMMARY OF THE INVENTION

Disclosed herein is a novel method for detecting non-therapeutic, therapeutic and toxic levels of aspirin in an individual which makes use of the urinary SA to SUA ratio. The use of a ratio (no units) for determining non-therapeutic, therapeutic and toxic levels of aspirin avoids the possibility of an error occurring during mathematical calculation through use of the wrong units or when recalculating the true salicylate concentration to account for sample dilution. Furthermore, the use of a urinary sample compared to a blood sample is less invasive and requires no sample preparation prior to analysis, such as hydrolysis of conjugated salicylic acid metabolites to form salicylic acid. Importantly, the SA to SUA ratio method can also be used at home by individuals on aspirin therapy, especially the elderly, to ensure an optimum level of aspirin is maintained and to avoid toxic levels. An increasing ratio of SA to SUA over time is indicative of increasing levels of aspirin that may be exceeding the optimum therapeutic levels and becoming toxic. Likewise, this method is appropriate for use in doctors' surgeries.

The SA to SUA ratio method for increasingly toxic levels of salicylic acid is guided, but not constrained, by the following values (Patel et al. 1990a & 1990b, Hutt et al. 1986, Ho et al. 1985, Rumbleet et al. 1980 and Gibson et al. 1975): SA:SUA between about 1.00:7.50 and about 1.00:3.00 indicates a therapeutic concentration, SA:SUA between about 1.00:3.00 and about 1.00:1.00 indicates a moderately toxic concentration, SA:SUA of greater than about 1.00:1.00 indicates a toxic concentration.

The invention also describes a method that uses a cut-off value for SUA in urine to signify a toxic concentration of aspirin or OD. This approach can be supported with multiple measurements of SUA over time to confirm SUA zero order kinetics at toxic aspirin levels or OD.

The invention further describes antibodies highly specific towards SUA.

A final aspect of the invention is a kit for determining toxic levels of aspirin in an in vitro urinary sample, comprising an antibody highly specific for SA and an antibody highly specific for SUA.

### Definitions

Aspirin—as aspirin is rapidly metabolised to salicylic acid which is the poisonous active component, any mention of aspirin in a toxic context implicitly refers to salicylic acid, unless otherwise qualified.

Toxic concentration—usually associated with an aspirin overdose event (the terms toxic concentration and OD are used interchangeably and refer to one or both of a single toxic ingestion event or to toxic levels induced by chronic ingestion, unless otherwise stated). The patient will usually have one or more concurrent symptoms such as nausea, vomiting and tinnitus. The general literature commonly describes a toxic concentration as aspirin ingestion of >300 mg/kg.

Moderately Toxic Concentration (MTC)—a concentration that exceeds the upper level of the recognised aspirin therapeutic range but is not an OD event. Chyka et al (2007) describe a MTC level as aspirin ingestion 150 mg/kg to 300 mg/kg or 6.5 g of aspirin equivalent.

A supra therapeutic concentration of aspirin refers to either a MTC or toxic concentration of aspirin.

Highly specific antibodies—antibodies that preferentially bind to a specific molecule. This preferential binding is verified through cross-reactivity (CR) studies which compares the binding of the antibody to the target molecule compared to structurally-related analogues and common drugs. The preferential binding of the antibodies to the target molecules compared to structurally-related analogues should be of such a magnitude so as not to compromise the value of the ratio and thus the integrity of the diagnosis. As can be seen in Table 2 of the Experimental and Results section, the structurally-related analogues of salicyluric acid each have an immeasurable CR value (<<1.00%) compared to salicyluric acid (100%) at their tested concentrations.

### DESCRIPTION OF DRAWINGS

FIG. 1 Synthesis of Hapten 1

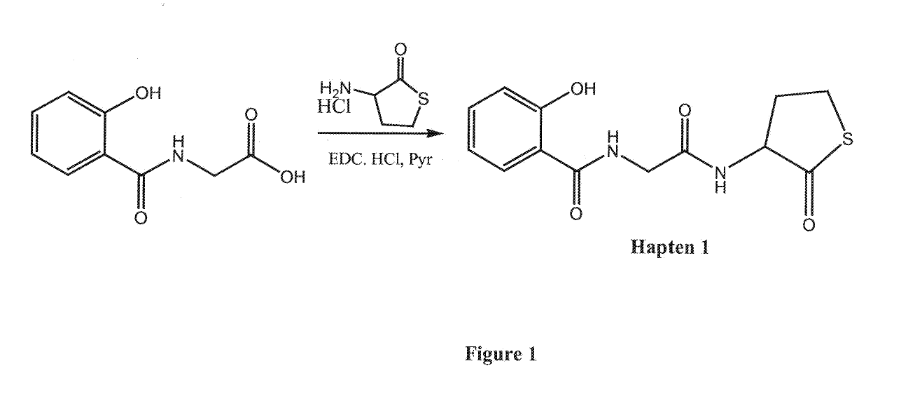

FIG. 2 Urinary salicylic acid (SA) to salicyluric acid (SUA) ratio as a function of increasing aspirin dose.

### DETAILED DESCRIPTION OF THE INVENTION

The invention describes the use of the SA:SUA ratio measured in an in vitro urinary sample of an individual to detect sub-therapeutic, therapeutic and toxic levels of aspirin in the individual. The invention also describes novel methods for detecting sub-therapeutic, therapeutic and toxic levels of aspirin in an individual using the urinary SA to SUA ratio. Preferably, it is used for detecting toxic levels of aspirin in an individual, especially overdose levels. The invention is especially useful for application in a testing device for use at home or at the doctor's surgery, to ensure that aspirin levels in individuals on aspirin therapy are at acceptable concentrations, and are not at sub-therapeutic or toxic concentrations. The therapeutic level of salicylic acid in blood of a healthy adult is generally considered to be 150-300 mg/l. This level corresponds to a urinary excretion ratio of SA to SUA of about 1:7.5. As the level of salicylic acid increases either due to chronic aspirin ingestion, or a single large dose, the urinary excretion concentration of SA increases relative to SUA.

As is known to the person skilled in the art, diagnostic assays based on biochemicals are affected by many factors such as the age, diet, sex, medical history, genotype etc., of the presenting patient. Thus, when establishing the ranges and cut-off levels attached to an assay these factors may be taken into consideration. A common approach to determining the optimal performance of a biochemical diagnostic assay is to use the ‘sensitivity’ and ‘specificity’ concept. The sensitivity is a measure of how good the test is at correctly identifying true positives, while the specificity is a measure of how good the test is at correctly identifying true negatives. From these values, the concepts of positive predictive value, negative predictive value and ROC curves can be used to assess the performance of the test. These and similar methods can be applied to the current invention.

The SA:SUA ratio method for increasingly toxic levels of aspirin is guided by the following literature values: less than about 1.00:3.00 indicates a non-toxic concentration, between about 1.00:3.00 and about 1.00:1.00 indicates a moderately toxic concentration, and greater than about 1.00:1.00 indicates a toxic concentration.

The SA:SUA ratio method for sub-therapeutic and therapeutic levels is guided by the following literature values: less than about 1.00:7.50 indicates a sub-therapeutic dose and between about 1.00:7.50 and about 1.00:3.00 indicates a therapeutic concentration.

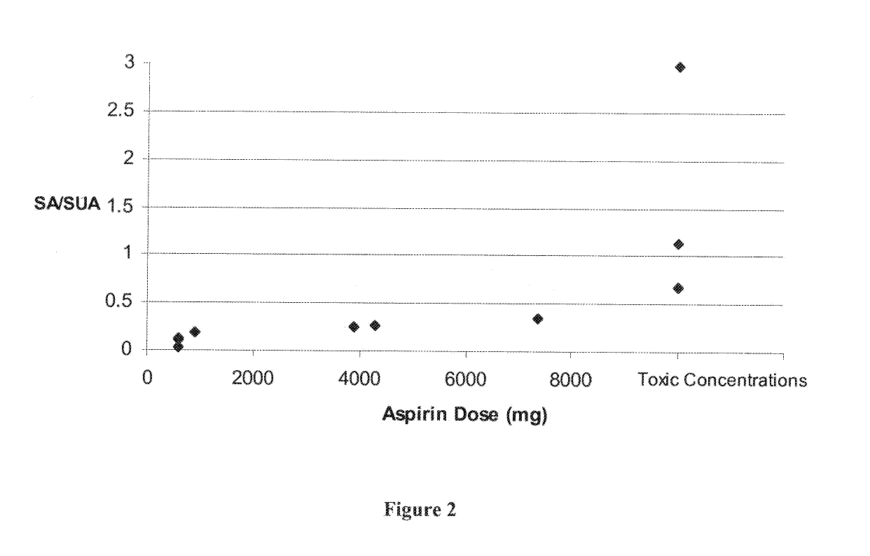

For practical purposes and for diagrammatical representation, these values are represented by the quotient of SA and SUA (FIG. 2). These values were derived from studies of patients who were undergoing aspirin therapy or who presented at accident and emergency departments having overdosed on aspirin (see Bibliography).

A first aspect of the invention is the use of the value of the SA:SUA ratio calculated from the levels of salicylic acid and salicyluric acid measured in an in vitro urinary sample of an individual as an indicator of sub-therapeutic, therapeutic, moderately toxic and toxic concentrations of aspirin in the individual. Preferably, the SA:SUA ratio is used as an indicator of a supra-therapeutic concentration of aspirin in an individual. More preferably, the SA:SUA ratio is used as an indicator of a toxic concentration of aspirin in an individual.

A second aspect of the invention is a method for determining a sub-therapeutic, a therapeutic, a moderately toxic or toxic concentration of aspirin in an individual by measuring the levels of salicylic acid and salicyluric acid in an in vitro urinary sample taken from the individual, calculating the ratio of SA:SUA in the sample, and comparing the calculated ratio to one or more ratios of salicylic acid to salicyluric acid indicative of a non-toxic concentration, a moderately toxic or toxic concentration of aspirin. Preferably the ratio of SA:SUA is equal to or less than about 1.00:7.50 indicates a sub-therapeutic concentration, between about 1.00:7.50 and about 1.00:3.00 indicates a therapeutic concentration, between about 1.00:3.00 and about 1.00:1.00 indicates a moderately toxic concentration, and greater than about 1.00:1.00 indicates a toxic concentration.

A third aspect of the invention is a method for determining a sub-therapeutic, a therapeutic, a moderately toxic or toxic concentration of aspirin in an individual by measuring the levels of salicylic acid and salicyluric acid in an in vitro urinary sample taken from the individual, calculating the ratio of SA:SUA in the sample, and comparing the calculated ratio to one or more ratios of salicylic acid to salicyluric acid indicative of a non-toxic concentration, a moderately toxic or toxic concentration of aspirin. Preferably the ratio of SA:SUA is equal to or less than 1.00:7.50 indicates a sub-therapeutic concentration, greater than 1.00:7.50 and up to 1.00:3.00 indicates a therapeutic concentration, greater than 1.00:3.00 and up to 1.00:1.00 indicates a moderately toxic concentration, and greater than 1.00:1.00 indicates a toxic concentration.

There are several possible analytical formats that could be used to measure the SA:SUA ratio such as HPLC and LC-MS. Preferably the format is an immunoassay as this enables portability and use outside of the clinical laboratory, unlike formats such as LC-MS. Immunoassay formats are varied and could be an ELISA or a lateral flow device, or the SA:SUA ratio test may be part of a multi-analyte array on a biochip or other suitable substrate. A combination of the SA:SUA test with other urine-based aspirin-related tests such as a 11-dehydro thromboxane B2 test for aspirin resistance, either incorporated in a portable device suitable for clinical or self-testing or on a biochip as part of a multi-analyte array for medium to high throughput testing, is an alternative application. Therefore, a further aspect of the invention describes the preferred analytical format that is used to execute the methods of the invention namely an immunoassay format comprising an antibody highly specific to salicylic acid and an antibody highly specific to salicyluric acid.

To enable the urinary SA:SUA ratio method to be used in an immunoassay format, antibodies highly specific to SUA and SA are required. It is well known to the skilled person that polyclonal antibodies generally have a wide-ranging specificity and recognise molecules with diverse epitopes. When a highly specific immunoassay is required it is standard practice to derive monoclonal antibodies from the original polyclonal pool. A monoclonal antibody highly specific towards SA has been described (Wang et al 2002). However, to the inventors' knowledge, antibodies highly specific towards SUA are unknown in the art. Antibodies described in commercial salicylate kits possess cross-reactivity to SUA, but also bind to several other aspirin metabolites such as SA and SPG. Attempts by the inventors to raise SUA-specific antibodies using what would be considered standard derivatisation routes to persons skilled in the art, using immunogens based on 4- and 5-derivatives of SUA e.g. 4-(BSA-CO—(CH₂)₃—NH-)SUA, failed. However, unexpectedly, an immunogen based on a 1-salicylic acid derivatisation produced a polyclonal antibody with high specificity to SUA (Table 2).

Therefore, a further aspect of the invention is an antibody highly specific to SUA raised through an immunogen formed from a salicyluric acid hapten, derivatised through the carboxy group of salicyluric acid, the immunogen being of the structure

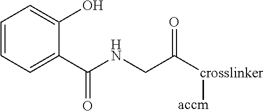

The crosslinker is well known in the art and is a group of atoms containing two or more functionalities capable of joining two or more discrete molecules. Functionalities in the context of the current invention imply reactive atoms or groups of atoms able to form bonds with other atoms or groups of atoms. Preferably the crosslinker is —X—Y— where X is a functionality, preferably O, N or S which attaches to the carbonyl group and Y is a C₁-C₁₀, more preferably a —C₂-C₆ substituted or unsubstituted straight chain alkylene moiety, arylene moiety or heterocyclic moiety which attaches to the accm by way of a second functionality. The accm, an antigenicity conferring carrier material, is a macromolecule such as a protein that confers immunogenicity to the non-immunogenic hapten. Accms are well known in the art but for the purpose of the current invention is preferably bovine thyroglobulin (BTG).

The invention also describes an antibody highly specific to salicyluric acid raised from an immunogen derived from the structure

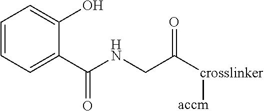

together with an antibody highly specific to salicylic acid, for use in an in vitro urinary immunoassay, to assess whether the level of aspirin in an individual is at a non-therapeutic, a therapeutic or a toxic concentration, where the crosslinker is —X—Y—, in which X is a functionality, preferably O, N or S that attaches to the carbonyl group and Y is a C₁-C₁₀, more preferably a —C₂-C₆ substituted or unsubstituted straight chain alkylene moiety, arylene moiety or heterocyclic moiety that attaches to the accm by way of a second functionality. The accm, an antigenicity conferring carrier material, is a macromolecule such as a protein that confers immunogenicity to the non-immunogenic hapten.

The highly specific antibody can be either a polyclonal or monoclonal antibody or a fragment thereof The method of monoclonal antibody selection from a polyclonal antibody is well known and commonly practised by persons skilled in the art.

The SUA-specific antibody enables, in conjunction with a SA-specific antibody, an immunoassay test for toxic levels of aspirin that uses the SA to SUA urinary ratio. The SUA-specific antibody also enables an immunoassay test for aspirin OD that uses a SUA urinary cut-off level, with multiple sampling over time to detect plateauing of SUA levels. This plateauing or levelling off is indicative of a saturation of the metabolic pathway leading to SUA. As SPG is also subject to Michaelis-Menten type kinetics, it should be amenable to the methods using SUA described herein.

The invention further discloses a kit for use in the in vitro determination of aspirin levels in a patient comprising an antibody specific for SA and an antibody specific for SUA. The kit may be in the form of, but is not limited to, a dip-stick device, a microfluidic device, antibodies for use in an ELISA, or a solid state device such as a biochip. When incorporated onto a biochip or for use in an ELISA format, the SA and SUA specific antibodies may be part of an antibody array for multi-analyte detection or determination, and may be used as part of a panel of antibodies to detect and determine aspirin-related analytes such as 11-dehydro thromboxane B2.

### EXAMPLE 1

### Conjugation of Salicyluric Acid to BSA

To a solution of salicyluric acid (22.05 mg, 0.113 mM) in DMF (1.0 ml) was added N,N-dicyclohexylcarbodiimide (DCC) (25.58 mg, 0.124 mM) and N-hydroxysuccinimide (NHS) (14.27 mg, 0.124 mM) and the mixture was stirred at room temperature overnight. The dicyclohexylurea formed was filtered and the solution obtained was added dropwise to a solution of BSA (150 mg, 2.3 82 M) in sodium bicarbonate solution (100 mM, 10 ml) pH 8.5 and the solution was incubated overnight at room temperature. The solution was then dialysed against 50 mM phosphate buffer pH 7.2 (3 changes) for 24 hours at 4° C., and freeze-dried. MALDI results showed 17 molecules of salicyluric acid had been conjugated to one molecule of BSA.

### EXAMPLE 2

### Conjugation of Salicyluric Acid to BTG

To a solution of salicyluric acid (26.34 mg, 0.135 mM) in DMF (1.0 ml) was added N,N-dicyclohexylcarbodiimide (DCC) (30.53 mg, 0.148 mM) and N-hydroxysuccinimide (NHS) (17.03 mg, 0.148 mM) and the mixture was stirred at room temperature overnight. The dicyclohexylurea formed was filtered and the solution obtained was added dropwise to a solution of BTG (150 mg) in sodium bicarbonate solution (100 mM, 10 ml) pH 8.5 and the solution was incubated overnight at room temperature. The solution was then dialysed against 50 mM phosphate buffer pH 7.2 (3 changes) for 24 hours at 4° C., and freeze-dried.

### EXAMPLE 3

### Preparation of Antisera

In order to generate polyclonal antisera, the immunogen (prepared in Example 2) was mixed with primary complete Freund's Adjuvant (Sigma) and injected into a host sheep to provide target-specific polyclonal antisera. On a monthly basis the immunogen (with incomplete Freund's Adjuvant) was injected into the sheep. The host animal was bled to yield a suitable volume of specific antiserum. IgG was then extracted from the antisera via Caprylic acid/ammonium sulphate precipitation of immunoglobulin.

### EXAMPLE 4

### Preparation of Hapten 1

To a solution of salicyluric Acid (1.95 g, 0.01 mol) in pyridine (25 ml) was added homocysteinethiolactone hydrochloride (1.54 g, 0.01 mol) and EDC hydrochloride (2.3 g, 0.012 mol) and the mixture was stirred at room temperature overnight. The solution was then concentrated to dryness and the crude product was purified by chromatography on silica gel using (95% chloroform/5% methanol) to give 1.7 g of Hapten 1 as a white solid. IR (λ: cm⁻¹): 3361.2, 32.81.3, 1696.5, 1665.4 and 1646.5; 13C NMR (δ: ppm): 205.6, 168.9, 159.9, 134.1, 128.7, 119.0, 117.6, 115.8, 58.5, 42.4, 30.4 and 27.0.

### EXAMPLE 5

### Conjugation of Hapten 1 to Maleimide HRP

Hapten 1 (2 mg) was dissolved a mixture of DMF/water (100 μl) and to this solution was added potassium hydroxide (2M) (10 μ\tilde{l} The mixture was allowed to stand for 10 minutes. Phosphate buffer (100 μl) was added to quench the reaction and the pH was adjusted to 7 by the addition of 0.1M HCl. This solution was added dropwise to maleimide modified HRP (20 mg) dissolved in phosphate buffer (1 ml) and the solution stirred at 4° C. overnight (protected from light). Excess hapten was removed with double PD-10 columns (Pharmacia) in series, pre-equilibrated with PBS at pH 7.2.

The Hapten 1—HRP conjugate was then dialysed with 10 L of PBS at pH 7.2 at 4° C.

### EXAMPLE 6

### Antibody Characterisation—Salicyluric Acid Dose-Response Curve and Cross-Reactivity Profile

Nine calibrator solutions of salicyluric acid (0-5.00 μg/ml) were prepared in calibrator buffer for standard curves. Cross-reactants (aspirin, 4-aminosalcylic acid, benzoic acid, diflusinal, gentisic acid, gentisuric acid, ibuprofen, methyl salicylate, salicylic acid and salsalate) were diluted in calibrator buffer (0-350 μg/ml). Assay diluent (155 μl), sample/antigen (25 μl), and diluted conjugate (Hapten 1-HRP 120 μl) were added to biochips wells. Biochips were incubated for 30 min at 30° C. in a thermoshaker at 370 rpm, and then washed with TBST buffer (2 quick washes followed by 4 washes with 2 min intervals). Biochips were developed with 250 μl luminol plus: peroxide (1:1) for 2 min in the dark, and were then imaged on the Randox Investigator (2×60 sec images).

TABLE 1
Results of biochip-based competitive immunoassay
using the ligands Hapten 1-HRP and salicyluric acid,
and the antibodies raised from SUA-BTG

| | SUA standard | | | |
|---|---|---|---|---|
| Level | (μg/ml) | Mean RLU | % CV | B/B0 |
| 1 | 0.00 | 13893 | 2 | 100 |
| 2 | 0.04 | 8448 | 3 | 61 |
| 3 | 0.08 | 6145 | 6 | 44 |
| 4 | 0.16 | 4443 | 4 | 32 |
| 5 | 0.31 | 2932 | 5 | 21 |
| 6 | 0.63 | 1616 | 2 | 12 |
| 7 | 1.25 | 928 | 7 | 7 |
| 8 | 2.50 | 451 | 2 | 3 |
| 9 | 5.00 | 204 | 7 | 1 |

RLU = relative light units;
CV = coefficient of variation;
B = absorbance at 450 nm at x μg/ml standard concentration,
B0 = absorbance at 450 nm at 0 μg/ml standard concentration;
IC₅₀(standard concentration which produces 50% B/B0) = 0.07 μg/ml.

TABLE 2
Cross-reactivity Data

| Compound | Concentration (μg/ml) | % Cross-reactivity |
|---|---|---|
| Salicyluric acid | 5 | 100 |
| Salicylic acid | 350 | <<1.00 |
| Hippuric acid | 350 | <<1.00 |
| Aspirin | 350 | <<1.00 |
| Gentisuric acid | 350 | <<1.00 |
| Methyl salicylate | 350 | <<1.00 |
| Diflusinal | 350 | <<1.00 |
| Ibuprofen | 350 | <<1.00 |
| Gentisic acid | 350 | <<1.00 |
| 4-aminosalicylic acid | 350 | <<1.00 |
| Benzoic acid | 350 | <<1.00 |

### BIBLIOGRAPHY

I. H. Hahn et al. 2000. Academic Emergency Medicine, 7: 1336-1337.
Goldfrank's Toxicologic Emergencies 2002. Editors Goldfrank, Flomenbaum, Lewin, Howland, Hoffman, Nelson. Seventh Edition. Published by McGraw-Hill.
G. F. O'Malley 2007. Emergency Medicine Clinics of North America, 25: 333-346.
S. Wang et al. 2002. An ELISA for the determination of salicylic acid implants using a monoclonal antibody. Plant Science, 162: 529-535.
D. L. Eldridge & C. P. Holstege 2006. Clinics in Laboratory Medicine, 26:13-3.
T. Gibson & G. Zaphiropoulus 1975. Kinetics of salicylate metabolism. Br. J. Clin. Pharmac., 2: 233-238.
R. H. Rumble et al. 1980. Metabolism of salicylate during chronic aspirin therapy. Br. J. Clin. Pharmac., 9: 41-45.
P. C. Ho et al. 1985. The effects of age and sex on the disposition of acetylsalicylic acid and its metabolites. Br. J. Clin. Pharmac., 19: 675-684.
A. J. Hutt et al. 1986. The metabolism of aspirin in man: a population study. Xenobiotica, 16: 239-249.
D. K. Patel et al. 1990a. Comparative metabolism of high doses of aspirin in man and rat. Xenobiotica, 20: 847-854.
D. K. Patel et al. 1990. Metabolism of aspirin after therapeutic and toxic doses. Hum. Exp. Toxicol., 9: 131-136.
W. Song and C. Dou 2003. One-step immunoassay for acetaminophen and salicylate in serum, plasma, and whole blood. J. Anal. Toxicol., 27: 366-371.
A. D. Fraser 1983. Clinical evaluation of the EMIT salicylic acid assay. Ther. Drug Monit., 5: 331-334
P. H. Chyka et al. 2007. Salicylate poisoning: an evidence-based consensus guideline for out of hospital management. Clin. Toxicol. (Phila), 45: 95-131.
S. L. Greene et al. 2005. Postgraduate Medical Journal, 81: 204-216

1-2. (canceled)

3. A method for determining a sub-therapeutic, a therapeutic, a moderately toxic or toxic concentration of aspirin in an individual by measuring the levels of salicylic acid and salicyluric acid in an in vitro urinary sample taken from the individual, calculating the ratio of salicylic acid to salicyluric acid in the sample, and comparing the calculated ratio to one or more ratios of salicylic acid to salicyluric acid indicative of a non-toxic concentration, a moderately toxic or toxic concentration of aspirin.

4. The method of claim 3 in which a ratio of salicylic acid to salicyluric acid equal to or less than about 1.00:3.00 indicates a sub-therapeutic or therapeutic concentration and a ratio greater than about 1.00:3.00 indicates a moderately toxic or toxic concentration.

5. The method of claim 3 in which a ratio of salicylic acid to salicyluric acid of greater than about 1.00:1.00 indicates a toxic concentration

6. The method of claim 3 in which a ratio of salicylic acid to salicyluric acid of less than about 1.00:7.50 indicates a sub-therapeutic concentration, between about 1.00:7.50 and about 1.00:3.00 indicates a therapeutic concentration, between about 1.00:3.00 and about 1.00:1.00 indicates a moderately toxic concentration, and greater than about 1.00:1.00 indicates a toxic concentration.

7. The method of claim 3 in which a ratio of salicylic acid to salicyluric acid equal to or less than 1.00:7.50 indicates a sub-therapeutic concentration, greater than 1.00:7.50 and up to 1.00:3.00 indicates a therapeutic concentration, greater than 1.00:3.00 and up to 1.00:1.00 indicates a moderately toxic concentration, and greater than 1.00:1.00 indicates a toxic concentration.

8. The method of claim 3 which is an immunoassay comprising an antibody highly specific to salicylic acid and an antibody highly specific to salicyluric acid.

9. An antibody highly specific to salicyluric acid raised from an immunogen of the following structure

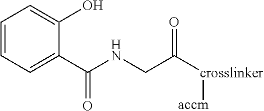

10. An antibody highly specific to salicyluric acid raised from an immunogen of the following structure 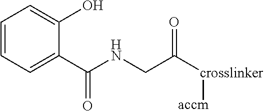
together with an antibody highly specific to salicylic acid, for use in an in vitro urinary immunoassay, to assess whether the level of aspirin in an individual is at a non-therapeutic, a therapeutic or a toxic concentration.

11. The antibody of claim 9 in which the crosslinker of the immunogen from which the antibody is raised is —X—Y—, where X is a heteroatom which attaches to the carbonyl group and Y is a C₁ to C₁₀ substituted or unsubstituted straight chain saturated alkylene moiety, or arylene moiety, or heterocyclic moiety which attaches to the accm.

12. A kit for detecting and determining therapeutic and toxic concentrations of aspirin in a patient comprising an antibody highly specific to salicyluric acid and an antibody highly specific to salicylic acid.

13. The antibody highly specific to salicyluric acid of claim 9 which is a polyclonal antibody.

14. The antibody of claim 9 in which the crosslinker of the immunogen from which the antibody is raised is —X—Y—, where X is a heteroatom which attaches to the carbonyl group and Y is a C₁ to C₁₀ substituted or unsubstituted straight chain saturated alkylene moiety, or arylene moiety, or heterocyclic moiety which attaches to the accm.

15. The antibody highly specific to salicyluric acid of claim 10 which is a polyclonal antibody.
